(19) United States
(12) Patent Application Publication
(10) Pub. No.: US 2007/0077218 A1
(43) Pub. Date: Apr. 5, 2007

(54) Composition containing a coloring agent

(75) Inventors: Aurelie Weinling, Paris (FR); Sandrine Champenois, Bagneux (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/508,857

(22) Filed: Aug. 24, 2006

Related U.S. Application Data
(60) Provisional application No. 60/712,406, filed on Aug. 31, 2005.

(30) Foreign Application Priority Data
Aug. 26, 2005 (FR) 05 52572

(51) Int. Cl. A61K 8/81

(52) U.S. Cl. 424/63; 424/70.11; 424/70.7

(57) ABSTRACT

The present invention relates to a composition preferably useful as a cosmetic eyeshadow containing a particular liquid fatty phase. The invention composition is characterized by its sheen. The invention also relates to a process for making up the eyelids using the composition.

### REFERENCE TO PRIOR APPLICATIONS

This application claims priority to U.S. provisional application 60/712,406 filed Aug. 31, 2005, and to French patent application 0552572 filed Aug. 26, 2005, both incorporated herein by reference.

### FIELD OF THE INVENTION

The present invention relates to a composition preferably useful as a cosmetic eyeshadow comprising a particular liquid fatty phase. The invention composition is characterized by its sheen. The invention also relates to a process for making up the eyelids using the composition.

In a preferred embodiment the invention relates to a composition comprising, in a cosmetically acceptable medium, at least one colouring agent chosen from diffractive pigments, interference pigments and reflective pigments, and mixtures thereof, a fatty phase comprising polydecene that is liquid at 25° C., at least one wax totally or partially in powder form and/or a polyurethane powder. Preferably the composition is one having a sheen of greater than or equal to 1000 gloss units.

### BACKGROUND OF THE INVENTION

Eyeshadows generally include a suitable vehicle and dyestuffs intended to give a certain colour effect to the makeup product and, after application, to the eyelids. They may be in water-based or anhydrous form.

Many makeup compositions of this type are in compact powder form generally comprising a fatty phase, conventionally known as a “binder”, and a pulverulent phase especially comprising colouring agents (pigments or nacres) and/or fillers. The main function of the binder is to ensure sufficient cohesion of the final composition, especially so as to prevent it from fragmenting, as may be caused by impacts, and moreover to make it easy to be taken up.

In point of fact, obtaining these properties moreover implies that the other components of the cosmetic composition, and in particular the fillers they contain, also prove to be suitable for a formulation of compact powder type.

It is sought to obtain eyeshadows that have a strong colour effect, in particular by incorporating a high content of pigments, in particular nacres.

However, as it turns out, these nacres generally precisely lack a good capacity to be compacted with standard binders and their presence in high content in an eyeshadow in compact powder form may significantly affect the cohesion of the resulting powder, or even, when they are in large amount, make it impossible to obtain a corresponding compact powder using a mechanical press.

The binder content then needs to be increased in order to improve the compacting, for example to scale of 10% by weight of binder in the composition, which generally proves to be detrimental to the surface aspect, which is then too greasy.

### OBJECTS OF THE INVENTION

One object of the invention is to satisfy the need for a cosmetic composition of compact powder type comprising large amounts of nacreous pigments and having a strong colour effect, while at the same time having satisfactory cohesion and erosion characteristics, and not otherwise being constraining in terms of industrial preparation. Other objects will become apparent upon a reading of the invention description in full.

### SUMMARY OF THE INVENTION

The inventors have found, surprisingly, that, among other things, a composition having the desired characteristics noted above is obtained by using, in an eyeshadow in compact powder form, particular colouring agents, in particular nacres, with a liquid fatty phase comprising polydecene that is liquid at 25° C. for example when combined with a wax, preferably in powder form, and/or a polyurethane powder.

This combination allows the production of a composition suitable for forming on keratin materials a film that has a strong colour effect and, in addition, the film of composition obtained is smooth and uniform and has strong sheen. The composition shows good stability on storage and does not become impaired over time when it suffers impacts; it also has a good capacity to be taken up.

### DETAILED DESCRIPTION OF THE PREFERRED EMBODIMENTS

More specifically, one subject of the invention is a composition, preferably one useful as an eyeshadow, in compact powder form comprising, typically in a cosmetically acceptable medium, a fatty phase comprising polydecene that is liquid at 25° C., at least one wax totally or partially in powder form and at least one colouring agent chosen from diffractive pigments, interference pigments and reflective particles, and mixtures thereof, the composition having a sheen at 45° of greater than or equal to 1000 gloss units.

As noted, the compositions of the invention are preferably useful as eyeshadows. Thus, for ease of reference, “eyeshadows” will be referred to below. Note, however, that the invention also relates to the described compositions in general whether or not they are useful as eyeshadows. In addiotn, it is typical, but not required, that they comprise a cosmetically acceptable medium.

According to a second aspect, a subject of the invention is an eyeshadow in compact powder form comprising, in a cosmetically acceptable medium, a fatty phase comprising polydecene that is liquid: at 25° C., at least one polyurethane powder and at least one colouring agent chosen from diffractive pigments, interference pigments and reflective particles, and mixtures thereof, the eyeshadow having a sheen at 45° of greater than or equal to 1000 gloss units.

According to a third aspect, a subject of the invention is an eyeshadow comprising, in a cosmetically acceptable medium, a fatty phase comprising polydecene that is liquid at 25° C. and at least 51% of at least one colouring agent chosen from diffractive pigments, interference pigments and reflective particles, and mixtures thereof.

A subject of the invention is also a cosmetic process for making up the eyelids, comprising the application to the eyelids of the eyeshadow as defined above.

A subject of the invention is also the use of an eyeshadow as defined above to obtain a shiny makeup, deposited on the eyelids.

Sheen

The sheen is measured according to the following protocol:

a) Preparation of the Sample

The eyeshadow in compacted form is taken up using a conventional sponge for application of compact powder, such as a piece of sponge of the type sold in the product “Photogenic” from Lancôme, which has been cut into shape beforehand using an 18 mm punch and bonded to the packaging cap of a lipstick using double-sided adhesive.

The stick comprising the sample is fixed onto an erosion machine (Ref. BIEN 010778) parametized in the following manner: applied force (adjusted using balance weights): 100 g; movement of the gears: position 7/7; number of rotations: 5. The eyeshadow sample is spread via the apparatus onto a piece of matt black contrast card (Leneta form WP1). covered with a 40 mm×70 mm Blenderm® patch (3M Sante). The eyeshadow deposit is 0.5 mg/cm² and is 0.35 mm thick.

b) Measurement of the Sheen

The sheen of the eyeshadow is evaluated by illuminating the sample, prepared according to point a), deposited on the contrast card, and measurement of the light intensity reflected by the sample as a function of the angle of observation, using a GON 360 spectrogoniophotometer from the company Instrument Systems.

The illumination angle is set at 45° and the measurement of the reflected intensity is read at 45°, with an integration time of 1 second.

The sheen value is expressed in gloss units.

The eyeshadow according to the invention preferably has a sheen at 45° of greater than or equal to 1000 gloss units, preferably greater than or equal to 2000 gloss units and better still greater than or equal to 2500 gloss units, which may be up to 10 000 gloss units.

Colouring Agent

The colouring agent is chosen from diffractive pigments, interference pigments and reflective pigments, and mixtures thereof. It may be present in the eyeshadow according to the invention in a content ranging from 40% to 90% by weight, preferably ranging from 50% to 80% by weight and better still from 55% to 70% by weight, in particular from 60% to 70% by weight, relative to the total weight of the composition.

In particular, the colouring agent is present in a content of greater than or equal to 51% by weight, preferably greater than or equal to 55% by weight, better still greater than or equal to 60% by weight and even better still greater than or equal to 65% by weight relative to the total weight of the composition.

Interference Pigment

The term “interference pigment” denotes a pigment capable of producing a colour via an interference phenomenon, for example between the light reflected by a plurality of superposed layers with different refractive indices, especially a succession of layers with high and low refractive indices.

An interference pigment may, for example, comprise more than four layers with different refractive indices.

The layers of the interference pigment may or may not surround a core, which may or may not have a flattened shape.

Nacres are examples of interference pigments.

Nacres

The term “nacre” should be understood as meaning coloured particles of any form, which may or may not be iridescent, especially produced by certain molluscs in their shell, or alternatively synthesized, and which have a colour effect via optical interference.

Examples of nacres that may be mentioned include nacreous pigments such as titanium mica coated with an iron oxide, mica coated with bismuth oxychloride, titanium mica coated with chromium oxide, titanium mica coated with an organic dye especially of the abovementioned type, and also nacreous pigments based on bismuth oxychloride. They may also be mica particles at the surface of which are superposed at least two successive layers of metal oxides and/or of organic dyestuffs.

The nacres may more particularly have a yellow, pink, red, bronze, orange, brown, gold and/or coppery colour or tint.

As illustrations of nacres that may be introduced as interference pigments into the first composition, mention may be made especially of the gold-coloured nacres sold especially by the company Engelhard under the name Brillant gold 212G (Timica), Gold 222C (Cloisonne), Sparkle gold (Timica), Gold 4504 (Chromalite) and Monarch gold 233X (Cloisonne); the bronze nacres sold especially by the company Merck under the name Bronze fine (17384) (colorona) and Bronze (17353) (Colorona) and by the company Engelhard under the name Super bronze (Cloisonne); the orange nacres sold especially by the company Engelhard under the name Orange 363C (Cloisonne) and Orange MCR 101 (Cosmica) and by the company Merck under the name Passion orange (Colorona) and Matte orange (17449) (Microna); the brown nacres sold especially by the company Engelhard under the name Nu-antique copper 340XB (Cloisonne) and Brown CL4509 (Chromalite); the nacres with a copper tint sold especially by the company Engelhard under the name Copper 340A (Timica); the nacres with a red tint sold especially by the company Merck under the name Sienna fine (17386) (Colorona); the nacres with a yellow tint sold especially by the company Engelhard under the name Yellow (4502) (Chromalite); the red nacres with a gold tint sold especially by the company Engelhard under the name Sunstone G012 (Gemtone); the pink nacres sold especially by the company Engelhard under the name Tan opale G005 (Gemtone); the black nacres with a gold tint sold especially by the company Engelhard under the name Nu antique bronze 240 AB (Timica), the blue nacres sold especially by the company Merck under the name Matte blue (17433) (Microna), the white nacres with a silvery tint sold especially by the company Merck under the name Xirona Silver, and the golden-green pink-orange nacres sold especially by the company Merck under the name Indian summer (Xirona), and mixtures thereof.

A dyestuff chosen from nacres, in particular micas covered with at least one layer of metal oxide, is preferably used.

Reflective Interference Particles

These particles may be chosen from particles with a synthetic substrate coated at least partially with at least one layer of at least one metal oxide, chosen, for example, from titanium oxide, especially TiO₂, iron oxide, especially Fe₂O₃, tin oxide, chromium oxide, barium sulfate and the following materials: MgF₂, CrF₃, ZnS, ZnSe, SiO₂, Al₂O₃, MgO, Y₂O₃, SeO₃, SiO, HfO₂, ZrO₂, CeO₂, Nb₂O₅, Ta₂O₅ and MOS₂, and mixtures or alloys thereof.

As examples of such particles, mention may be made, for example, of particles comprising a synthetic mica substrate coated with titanium dioxide, or glass particles coated with either brown iron oxide, titanium oxide, tin oxide or a mixture thereof, for instance those sold under the brand name Reflecks® by the company Engelhard.

Goniochromatic Pigment

For the purposes of the present invention, the term “goniochromatic pigment” means a colouring agent for obtaining, when the composition is spread onto a support, a colour trajectory in the plane a*b* of the CIE 1976 calorimetric space that corresponds to a variation Dh° of the angle of hue h° of at least 20° when the angle of observation is varied relative to the normal between 0° and 80°, for an angle of light incidence of 45°.

The colour trajectory may be measured, for example, using an Instrument Systems brand spectrogonio-reflectometer of reference GON 360 Goniometer, after the second composition has been spread in fluid form to a thickness of 300 μm using an automatic spreader onto an Erichsen brand contrast card of reference Typ 24/5, the measurement being taken on the black background of the card.

The goniochromatic pigment may be chosen, for example, from multilayer interference structures and liquid-crystal colouring agents.

In the case of a multilayer structure, it may comprise, for example, at least two layers, each layer being made, for example, from at least one material chosen from the group consisting of the following materials: MgF₂, CeF₃, ZnS, ZnSe, Si, SiO₂, Ge, Te, Fe₂O₃, Pt, Va, Al₂O₃, MgO, Y₂O₃, S₂O₃, SiO, HfO₂, ZrO₂, CeO₂, Nb₂O₅, Ta₂O₅, TiO₂, Ag, Al, Au, Cu, Rb, Ti, Ta, W, Zn, MOS₂, cryolite, alloys and polymers, and combinations thereof.

The multilayer structure may or may not have, relative to a central layer, symmetry regarding the chemical nature of the stacked layers. Different effects are obtained depending on the thickness and the nature of the various layers.

Examples of symmetrical multilayer interference structures are, for example, the following structures: Fe₂O₃/SiO₂/Fe₂O₃/SiO₂/Fe₂O₃, a pigment having this structure being sold under the name Sicopearl by the company BASF; MOS₂/SiO₂/mica-oxide/SiO₂/MOS₂; Fe₂O₃/SiO₂/mica-oxide/SiO₂/Fe₂O₃; TiO₂/SiO₂/TiO₂ and TiO₂/Al₂O₃/TiO₂, pigments having these structures being sold under the name Xirona by the company Merck (Darmstadt).

The liquid-crystal colouring agents comprise, for example, silicones or cellulose ethers on which are grafted mesomorphic groups. Liquid-crystal goniochromatic particles that may be used, for example, are those sold by the company Chenix and also those sold under the name Helicone® HC by the company Wacker.

Goniochromatic pigments that may also be used include certain nacres, pigments with effects on synthetic substrate, especially a substrate of alumina, silica, borosilicate, iron oxide or aluminium type, or interference holographic flakes derived from a polyterephthalate film.

The material may also comprise dispersed goniochromatic fibres. Such fibres may be less than 80 μm long, for example.

Diffractive Pigment

For the purposes of the present invention, the term “diffractive pigment” denotes a pigment capable of producing a colour variation according to the angle of observation when lit with white light, on account of the presence of a structure that diffracts light. Such a pigment is also occasionally known as a holographic pigment.

A diffractive pigment may comprise a diffracting network capable, for example, of diffracting an incident monochromatic light ray in defined directions.

The diffraction network may comprise a periodic unit, especially a line, the distance between two adjacent units being of the same order of magnitude as the wavelength of the incident light.

When the incident light is polychromatic, the diffraction network will separate the various spectral components of the light and produce a rainbow effect.

Reference may appropriately be made regarding the structure of diffractive pigments to the article “Pigments Exhibiting Diffractive Effects” by Alberto Argoitia and Matt Witzman, 2002, Society of Vacuum coaters, 45^{th} Annual Technical Conference Proceedings 2002.

The diffractive pigment may be made with units having different profiles, especially triangular, symmetrical or non-symmetrical, in gaps, of constant or non-constant width, sinusoidal, in ladder form.

The spatial frequency of the network and the depth of the units will be chosen as a function of the degree of separation of the various orders desired. The frequency may range, for example, between 500 and 3000 lines per mm.

Preferably, the particles of the diffractive pigment each have a flattened form, and are especially in the form of platelets.

The same pigment particle may comprise two crossed, perpendicular or non-perpendicular diffraction networks, of identical or different ruling.

The diffractive pigment may have a multilayer structure comprising a layer of a reflective material, covered at least on one side with a layer of a dielectric material. The latter material may give the diffractive pigment better rigidity and durability. The dielectric material may thus be chosen, for example, from the following materials: MgF₂, SiO₂, Al₂O₃, AlF₃, CeF₃, LaF₃, NdF₃, SmF₂, BaF₂, CaF₂, LiF and combinations thereof. The reflective material may be chosen, for example, from metals and alloys thereof, and also from non-metallic reflective materials. Among the metals that may be used, mention may be made of Al, Ag, Cu, Au, Pt, Sn, Ti, Pd, Ni, Co, Rd, Nb, Cr and Fe, and materials, combinations or alloys thereof, and doping products thereof with rare-earth metals.

Such a reflective material may, by itself, constitute the diffractive pigment, which will then be monolayer.

As a variant, the diffractive pigment may comprise a multilayer structure comprising a core of a dielectric material covered with a reflective layer at least on one side, or even totally encapsulating the core. A layer of a dielectric material may also cover the reflective layer(s). The dielectric material used is then preferably mineral, and may be chosen, for example, from metal fluorides, metal oxides, metal sulfides, metal nitrides, and metal carbides, and combinations thereof. The dielectric material may be in crystalline, semi-crystalline or amorphous form. In this configuration, the dielectric material may be chosen, for example, from the following materials: MgF₂, SiO, SiO₂, Al₂O₃, TiO₂, WO, AlN, BN, B₄C, WC, TiC, TiN, N₄Si₃, ZnS, glass particles and carbons of diamond type, and combinations thereof.

As a variant, the diffractive pigment may be composed of a preformed dielectric or ceramic material such as a mineral in natural leaflet form, for example mica perovskite or talc, synthetic leaflets formed from glass, alumina, SiO₂, carbon, an iron oxide/mica, mica coated with BN, BC, graphite or bismuth oxychloride, and combinations thereof.

Instead of a layer of a dielectric material, other materials that improve the mechanical properties may be suitable for use. Such materials may comprise silicone, metal silicides, semiconductive materials formed from elements of groups III, IV and V, metals with a cubic-centred crystal structure, cermet compositions or materials and semiconductive glasses, and various combinations thereof.

The diffractive pigment used may be chosen especially from those described in the American patent application U.S. 2003/0 031 870 published on 13 Feb. 2003.

A diffractive pigment may comprise, for example, the following structure: MgF₂/Al/MgF₂, a diffractive pigment having this structure being sold under the name Spectraflair 1400 Pigment Silver by the company Flex Products, or Spectraflair 1400 Pigment Silver FG. The weight proportion of MgF₂ may be between 80% and 95% of the total weight of the pigment.

Other diffractive pigments are sold under the names Metalure® Prismatic by the company Eckart.

Other possible structures are Fe/Al/Fe or Al/Fe/Al.

The size of the diffractive pigment may be, for example, between 5 and 200 μm and better still between 5 and 100 μm, for example between 5 and 30 μm.

The thickness of the diffractive pigment particles may be less than or equal to 3 μm and better still 2 μm, for example about 1 μm.

Pigments or Reflective Particles

The term “reflective particles” denotes particles whose size, structure, especially the thickness of the layer(s) of which they are made and of their physical and chemical nature, and surface state allow them to reflect incident light. This reflection may, where appropriate, have an intensity sufficient to create at the surface of the composition or of the mixture, when it is applied to the support to be made up, points of overbrightness that are visible to the naked eye, i.e. more luminous points that contrast with their environment by appearing to sparkle.

The reflective particles may be selected so as not to significantly alter the coloration effect generated by the colouring agents with which they are combined, and more particularly so as to optimize this effect in terms of colour yield. They may more particularly have a yellow, pink, red, bronze, orange, brown, gold and/or coppery colour or tint.

These particles may have varied forms and may especially be in platelet or globular form, in particular spherical.

Irrespective of their form, the reflective particles may or may not have a multilayer structure, and, in the case of a multilayer structure, for example at least one layer of uniform thickness, especially a reflective material.

When the reflective particles do not have a multilayer structure, they may be composed, for example, of metal oxides, especially titanium or iron oxides obtained via synthesis.

When the reflective particles have a multilayer structure, they may comprise, for example, a natural or synthetic substrate, especially a synthetic substrate at least partially coated with at least one layer of a reflective material, especially of at least one metal or metallic material. The substrate may be a monomaterial, multimaterial, organic and/or mineral substrate.

More particularly, it may be chosen from glasses, ceramics, graphite, metal oxides, aluminas, silicas, silicates, especially aluminosilicates and borosilicates, and synthetic mica, and mixtures thereof, this list not being limiting.

The reflective material may comprise a layer of metal or of a metallic material.

Reflective particles are described especially in documents JP-A-09188830, JP-A-10158450, JP-A-10158541, JP-A-07258460 and JP-A-05017710.

Again as an example of reflective particles comprising a mineral substrate coated with a layer of metal, mention may also be made of particles comprising a silver-coated borosilicate substrate.

Particles with a silver-coated glass substrate, in the form of platelets, are sold under the name Microglass Metashine REFSX 2025 PS by the company Toyal. Particles with a glass substrate coated with nickel/chromium/molybdenum alloy are sold under the name Crystal Star GF 550 and GF 2525 by this same company.

Particles comprising a metallic substrate such as silver, aluminium, iron, chromium, nickel, molybdenum, gold, copper, zinc, tin, manganese, steel, bronze or titanium, may also be used, the substrate being coated with at least one layer of at least one metal oxide such as titanium oxide, aluminium oxide, iron oxide, cerium oxide, chromium oxide or silicon oxides, and mixtures thereof.

Examples that may be mentioned include aluminium powder, bronze powder or copper powder coated with SiO₂ sold under the name Visionaire by the company Eckart.

Liquid Fatty Phase

The liquid fatty phase present in the composition according to the invention comprises polydecene that is liquid at 25° C. The polydecene may especially be hydrogenated polydecene.

The polydecene advantageously has a weight-average molecular weight ranging from 800 to 3000, preferably ranging from 1000 to 2500 and preferentially ranging from 1200 to 2200. Such a polydecene allows the preparation of a compact and composition that shows good impact strength.

Polydecenes that may be used include those sold under the names:
Ceraflow E by the company Shamrock, with a weight-average molecular weight of about 1400,
Ceraflow HE by the company Shamrock, with a weight-average molecular weight of about 2000.

The polydecene that is liquid at 25° C. may be present in the composition according to the invention in a content ranging from 1% to 20% by weight, preferably ranging from 2% to 15% by weight and preferentially ranging from 3% to 10% by weight relative to the total weight of the composition.

The fatty phase of the composition according to the invention may comprise an additional oil. The additional oil may be chosen from the oils conventionally used as binder in compact powders. Among the additional oils that may be used, mention may be made of soybean oil, grapeseed oil, sesame seed oil, corn oil, rapeseed oil, sunflower oil, cottonseed oil, avocado oil, olive oil, castor oil, jojoba oil or groundnut oil; hydrocarbon oils such as liquid paraffin, squalane or petroleum jelly; fatty esters such as isopropyl myristate, isopropyl palmitate, butyl stearate, isodecyl stearate, hexyl laurate, isononyl isononanoate, 2-ethylhexyl palmitate, 2-hexyldecyl laurate, 2-octyldecyl palmitate, 2-octyldodecyl myristate or lactate, 2-diethylhexyl succinate, diisostearyl malate, glyceryl or diglyceryl triisostearate and glyceryl triethyl hexanoate; silicone oils such as polymethylsiloxanes, polymethylphenylsiloxanes, polysiloxanes modified with fatty acids, fatty alcohols or polyoxyalkylenes, fluoro silicones and perfluoro oils; higher fatty acids such as myristic acid, palmitic acid, stearic acid, behenic acid, oleic acid, linoleic acid, linolenic acid or isostearic acid; higher fatty alcohols such as cetanol, stearyl alcohol or oleyl alcohol.

Wax

According to one particular variant of the invention, the eyeshadow comprises at least one wax that is totally or partially in the form of powder, especially micronized powder, to facilitate its use in the preparation of the cosmetic composition.

For the purposes of the present invention, the term “wax” means a lipophilic fatty compound, which is solid at room temperature (25° C.) and atmospheric pressure (760 mmHg, i.e. 10⁵ Pa) which undergoes a reversible solid/liquid change of state, and which in particular has a melting point of greater than or equal to 30° C., especially greater than or equal to 55° C., which may be up to 250° C., especially up to 230° C. and in particular up to 120° C.

By bringing the wax to its melting point, it is possible to make it miscible with the oils and to form a microscopically homogeneous mixture, but on returning the temperature of the mixture to room temperature, recrystallization of the wax in the oils of the mixture is obtained.

According to the invention, the melting point values correspond to the melting peak measured using a differential scanning calorimeter (DSC), for example the calorimeter sold under the name DSC 30 by the company Mettler, with a temperature rise of 5 or 10° C. per minute.

For the purposes of the invention, the waxes include those generally used in cosmetics or dermatology. They may especially be hydrocarbon-based waxes, silicone waxes and/or fluoro waxes, optionally comprising ester or hydroxyl functions. They may also be of natural or synthetic origin.

Non-limiting illustrations of waxes that may especially be mentioned include:
beeswax, lanolin wax and Chinese insect waxes; rice wax, carnauba wax, candelilla wax, ouricury wax, cork fibre wax, sugarcane wax, Japan wax and sumach wax; montan wax; microcrystalline waxes, paraffin waxes, ozokerites, ceresin wax, lignite wax, polyethylene waxes, the waxes obtained by Fisher-Tropsch synthesis, and fatty acid esters and glycerides that are solid at 40° C. and especially above 55° C.,
the waxes obtained by catalytic hydrogenation of animal or plant oils containing linear or branched C₈-C₃₂ fatty chains, especially hydrogenated jojoba oil, hydrogenated sunflower oil, hydrogenated castor oil, hydrogenated coconut oil and hydrogenated lanolin oil,
silicone waxes or fluoro waxes, and
mixtures thereof.

Among the waxes that may be used in powder form, mention may be made especially of the carnauba wax microbeads sold under the name MicroCare 350® by the company Micro Powders and the paraffin wax microbeads sold under the name MicroEase 114S® by the company Micro Powders.

The wax that is totally or partially in powder form may be present in a content ranging from 0.1% to 45% by weight, preferably from 0.5% to 20% by weight and better still from 1% to 10% by weight relative to the total weight of the composition.

Solid Fatty Phase

The composition according to the invention comprises, besides the wax in powder form, at least one fatty phase comprising at least one solid fatty phase, also known as a “solid binder”.

The purposes of the present invention, the term “solid binder” means a fatty phase whose melting point may be greater than or equal to 30° C. and may especially range from 30 to 250° C. and in particular from 30 to 230° C. This solid fatty phase may comprise at least one compound chosen from the additional waxes, which are not partially or totally in powder form, and metal soaps, and mixtures thereof.

The additional waxes may be chosen from the waxes described above.

Metal soaps that may especially be mentioned include the metal soaps of fatty acids containing from 12 to 22 carbon atoms and in particular those containing from 12 to 18 carbon atoms.

The metal of the metal soap may especially be zinc or magnesium.

The fatty acid may especially be chosen from lauric acid, myristic acid, stearic acid and palmitic acid.

Metal soaps that may be used include zinc laurate, magnesium stearate, magnesium myristate and zinc stearate, and mixtures thereof.
metal soaps derived from organic carboxylic acids containing from 8 to 22 carbon atoms and preferably from 12 to 18 carbon atoms, for example zinc stearate, magnesium stearate, lithium stearate, zinc laurate or magnesium myristate; these soaps are generally present in the form of particles less than 10 μm in size;

According to one particular variant of the invention, the solid fatty phase may comprise at least one metal soap that is totally or partially present in powder form.

The composition according to the invention may comprise at least one solid fatty phase in a content ranging from 0.5% to 45% by weight,. especially from 1% to 20% by weight and in particular from 1% to 10% by weight relative to the total weight of the composition.

Fillers

The eyeshadow according to the invention may comprise at least one filler that may be organic or mineral, and of spherical or lamellar form.

The incompactable filler may be organic or mineral, and of spherical or lamellar form.

Among the incompactable fillers of spherical form that may be mentioned are:
silica microspheres, especially of open porosity or, preferably, hollow silica microspheres, such as the products “Silica Beads SB 700/HA” or “Silica Beads SB 700” from the company Maprecos; these microspheres may be impregnated with a cosmetic active agent;
microporous polymer microspheres, which have a structure similar to that of a sponge; they generally have a specific surface area of at least 0.5 m²/g and in particular of at least 1 m²/g, the specific surface area having no upper limit other than that resulting from the practical possibility of making microspheres of very high porosity: the specific surface area may be, for example, up to 1000 m²/g or even more. Illustrations of these microspheres that may be mentioned include acrylic polymer microspheres, such as those made of crosslinked acrylate copolymer “Polytrap 6603 Adsorber” from the company RP Scherer, and those made of polymethyl methacrylate “Micropearl M 100” from the company SEPPIC;
polyurethane powder, such as the powdered copolymer of hexamethylene diisocyanate and of trimethylol hexyl lactone sold under the names Plastic Powder D-400 and T-7 by the company Toshiki;
polymer microcapsules bearing only one closed cavity and form a reservoir, which may contain a liquid, especially a cosmetic active agent; they are prepared via known processes, such as those described in patents U.S. Pat. No. 3,615,972 and EP-A 0 56 219. They may be made, for example, of polymers or copolymers of ethylenically unsaturated acid, amine or ester monomers, of urea-formaldehyde polymers or of vinylidene chloride polymers or copolymers; by way of example, mention may be made of microcapsules made of methyl acrylate or methacrylate polymers or copolymers, or alternatively of copolymers of vinylidene chloride and of acrylonitrile; among these polymers, mention will be made especially of those containing 20-60% by weight of units derived from vinylidene chloride, 20-60% by weight of units derived from acrylonitrile and 0-40% by weight of other units such as units derived from an acrylic and/or styrene monomer; crosslinked acrylic polymers or copolymers may also be used;
elastomeric crosslinked organopolysiloxane spherical powders, described especially in document JP-A-02 243 612, such as those sold under the name “Trefil Powder E-506C” by the company Dow Corning.

According to one particular embodiment, the eyeshadow according to the invention comprises a polyurethane powder, for instance the powdered copolymer of hexamethylene diisocyanate and of trimethylol hexyl lactone sold under the names Plastic Powder D-400 and T-7 by the company Toshiki.

The incompactable filler may be present in a content ranging from 0.1% to 45% by weight, especially from 0.5% to 20% by weight and in particular from 1% to 10% by weight relative to the total weight of the composition.

Compatible fillers of lamellar form that may be mentioned include:
talcs or hydrated magnesium silicates, especially in the form of particles generally less than 40 μm in size;
micas or aluminosilicates of varied composition that are especially in the form of flakes from 2 to 200 μm and preferably 5-70 μm in size and from 0.1 to 5 μm and preferably 0.2-3 μm in thickness, these micas possibly being of natural origin (for example muscovite, margarite, roscoelite, lipidolite or biotite) or of synthetic origin;
clays such as sericites, which belong to the same chemical and crystalline class as muscovite;
kaolin or hydrated aluminium silicate, which is especially in the form of particles of isotropic forms generally less than 30 μm in size;
boron nitrides.

Among the compactable fillers of organic lamellar type that may be mentioned are powders of tetrafluoroethylene polymers, such as “Ceridust 9205 F” from the company Clariant.

Compactable fillers of spherical form that may be mentioned include:
zinc oxides and titanium oxides, generally used in the form of particles not exceeding a few micrometres in size (or even less than 1 μm in the case of titanium oxide);
precipitated calcium carbonate, especially in the form of particles greater than 10 μm in size;
magnesium carbonate and magnesium hydrogen carbonate;
hydroxyapatite;
powders of non-expanded synthetic polymers, such as polyethylene, polyesters (for example polyethylene isophthalate or terephthalate) and polyamides (for example Nylon), in the form of particles less than 50 μm in size;
powders of spheronized, crosslinked or non-crosslinked synthetic polymers, for instance polyamide powders such as poly-β-alanine powder or Nylon powder, for example “Orgasol” powder from the company Atochem, polyacrylic acid or polymethacrylic acid powder, powders of polystyrene crosslinked with divinylbenzene, and silicone resin powders, and
powders of organic materials of natural origin, for instance starches, especially corn starch, wheat starch or rice starch.

The compactable fillers may be present in a content ranging from 0.05% to 40% by weight and preferably ranging from 0.1% to 20% by weight relative to the total weight of the composition.

The composition according to the invention may comprise, besides the colouring agent chosen from diffractive pigments, interference pigments and reflective particles, at least one “additional” colouring agent, producing a colour by absorption of at least part of the visible spectrum.

A colour produced by absorption of light is also occasionally termed a chemical colour, as opposed to the colours produced by an interference phenomenon, including diffraction, which are also referred to as physical colours. The phenomenon of absorption of light energy by the colouring agent may be based on electronic transitions.

The colouring agent that produces a colour by an absorption phenomenon may consist of an organic or mineral or hybrid pigment comprising both organic material and mineral material.

The colouring agent may be a particulate or non-particulate compound.

When the colouring agent comprises a dye, this dye may be chosen from liposoluble and water-soluble dyes.

The liposoluble dyes are, for example, Sudan red, DC Red 17, DC Green 6, β-carotene, soybean oil, Sudan brown, DC Yellow 11, DC Violet 2, DC Orange 5 and quinoline yellow.

The water-soluble dyes are, for example, beetroot juice and methylene blue.

The dyes may represent, for example, from 0.1% to 20% or even from 0.1% to 6% of the weight of the composition, when present.

The colouring agent may also be an organic pigment or lake chosen from the materials below, and mixtures thereof:
cochineal carmine,
organic pigments of azo, anthraquinone, indigoid, xanthene, pyrene, quinoline, triphenylmethane or fluorane dyes,
organic lakes or insoluble sodium, potassium, calcium, barium, aluminium, zirconium, strontium or titanium salts of acidic dyes such as azo, anthraquinone, indigoid, xanthene, pyrene, quinoline, triphenylmethane or fluorane dyes, these dyes possibly comprising at least one carboxylic or sulfonic acid group.

Among the organic pigments that may especially be mentioned are those known under the following names: D&C Blue No. 4, D&C Brown No. 1, D&C Green No. 5, D&C Green No. 6, D&C Orange No. 4, D&C Orange No. 5, D&C Orange No. 10, D&C Orange No. 11, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, D&C Violet No. 2, D&C Yellow No. 7, D&C Yellow No. 8, D&C Yellow No. 10, D&C Yellow No. 11, FD&C Blue No. 1, FD&C Green No. 3, FD&C Red No. 40, FD&C Yellow No. 5, FD&C Yellow No. 6.

The colouring agent may be an organic lake supported on an organic support such as rosin or aluminium benzoate, for example.

Among the organic lakes that may be mentioned in particular are those known under the following names: D&C Red No. 2 Aluminium lake, D&C Red No. 3 Aluminium lake, D&C Red No. 4 Aluminium lake, D&C Red No. 6 Aluminium lake, D&C Red No. 6 Barium lake, D&C Red No. 6 Barium/Strontium lake, D&C Red No. 6 Strontium lake, D&C Red No. 6 Potassium lake, D&C Red No. 7 Aluminium lake, D&C Red No. 7 Barium lake, D&C Red No. 7 Calcium lake, D&C Red No. 7 Calcium/Strontium lake, D&C Red No. 7 Zirconium lake, D&C Red No. 8 Sodium lake, D&C Red No. 9 Aluminium lake, D&C Red No. 9 Barium lake, D&C Red No. 9 Barium/Strontium lake, D&C Red No. 9 Zirconium lake, D&C Red No. 10 Sodium lake, D&C Red No. 19 Aluminium lake, D&C Red No. 19 Barium lake, D&C Red No. 19 Zirconium lake, D&C Red No. 21 Aluminium lake, D&C Red No. 21 Zirconium lake, D&C Red No. 22 Aluminium lake, D&C Red No. 27 Aluminium lake, D&C Red No. 27 Aluminium/Titanium/Zirconium lake, D&C Red No. 27 Barium lake, D&C Red No. 27 Calcium lake, D&C Red No. 27 Zirconium lake, D&C Red No. 28 Aluminium lake, D&C Red. No. 30 lake, D&C Red No. 31 Calcium lake, D&C Red No. 33 Aluminium lake, D&C Red No. 34 Calcium lake, D&C Red No. 36 lake, D&C Red No. 40 Aluminium lake, D&C Blue No. 1 Aluminium lake, D&C Green No. 3 Aluminium lake, D&C Orange No. 4 Aluminium lake, D&C Orange No. 5 Aluminium lake, D&C Orange No. 5 Zirconium lake, D&C Orange No. 10 Aluminium lake, D&C Orange No. 17 Barium lake, D&C Yellow No. 5 Aluminium lake, D&C Yellow No. 5 Zirconium lake, D&C Yellow No. 6 Aluminium lake, D&C Yellow No. 7 Zirconium lake, D&C Yellow No. 10 Aluminium lake, FD&C Blue No. 1 Aluminium lake, FD&C Red No. 4 Aluminium lake, FD&C Red No. 40 Aluminium lake, FD&C Yellow No. 5 Aluminium lake, FD&C Yellow No. 6 Aluminium lake.

The chemical materials corresponding to each of the organic dyestuffs mentioned above are mentioned in the publication “International Cosmetic Ingredient Dictionary and Handbook”, 1997 edition, pages 371 to 386 and 524 to 528, published by “The Cosmetic, Toiletry, and Fragrance Association”, the content of which is incorporated into the present patent application by reference.

The inorganic or mineral pigments are for example
iron oxide, titanium oxide, zirconium oxide, cerium oxide, zinc oxide, iron oxide or chromium oxide,
ferric blue, manganese violet, ultramarine blue, pink or violet, chromium hydrate, chromium hydroxide or bismuth oxychloride,
and mixtures thereof.

The colouring agent may be a composite pigment, comprising a core at least partially coated with a shell.

Composite Pigments

A composite pigment according to the invention may be composed especially of particles comprising:
a mineral core,
at least one at least partial coating of at least one organic dyestuff.

At least one binder may advantageously contribute to the fixing of the organic dyestuff to the mineral core.

The composite pigment particles may have varied forms. These particles may especially be in platelet or globular form, in particular spherical, and may be hollow or solid. The term “platelet form” denotes particles for which the ratio of the largest size to the thickness is greater than or equal to 5.

A composite pigment may have, for example, a specific surface area of between 1 and 1000 m²/g, especially between 10 and 600 m²/g approximately and in particular between 20 and 400 m²/g approximately. The specific surface area is the value measured by the BET method.

The mass proportion of the core may exceed 50% and may range, for example, from 50% to 70%, for example from 60% to 70%, relative to the total weight of the composite pigment.

The eyeshadow according to the invention may comprise at least one filler that may be organic or mineral, of spherical or lamellar form.

The composite pigment may be made, for example, via one of the processes described in European patent applications EP 1 184 426 and EP 1 217 046, the content of which is incorporated herein by reference, advantageously via the process described in patent application EP 1 184 426.

Another method for manufacturing a composite pigment is described in patent JP 3286463, which discloses a solution precipitation process.

According to one embodiment, the dyestuff that produces a colour by absorption of at least part of the visible spectrum is present in the composition according to the invention in a content of less than or equal to 10% by weight and preferably less than or equal to 5% by weight.

Accordingly, a subject of the invention is also an eyeshadow comprising, in a cosmetically acceptable medium, a fatty phase comprising polydecene that is liquid at 25° C., the composition comprising less than 10% of colouring agent that produces a colour by absorption of at least part of the visible spectrum.

The composition according to the invention may be an anhydrous composition, i.e. a composition containing less than 2% by weight of water, or even less than 0.5% water, especially less than 0.2% water, the water not being added during the preparation of the composition, but corresponding to the residual water provided by the mixed ingredients.

The composition according to the invention may comprise at least one cosmetic additive chosen from the group formed by preserving agents, fragrances, vitamins, moisturizers, softeners, sunscreens, film-forming polymers, sequestering agents and acidifying or basifying agents.

Needless to say, a person skilled in the art will take care to select this or these optional additional compound(s), and/or the amount thereof, such that the advantageous properties of the composition according to the invention are not, or are not substantially, adversely affected by the envisaged addition.

The composition may be prepared by mixing together the ingredients of the pulverulent phase (fillers and pigments), followed by adding the fatty phase with stirring, the mixture then being ground and/or mixed vigorously, optionally screened, and then poured into a dish and compacted, for example using a press.

The composition thus obtained is in the form of a compact powder.

The present invention is illustrated in greater detail in the examples that follow.

### EXAMPLE 1

The following compacted eyeshadow was prepared:

| | |
|---|---|
| Mica/black iron oxide | 70 g |
| (Microna Matte Black from Merck) | |
| Black iron oxides | 4 g |
| Talc | 0.2 g |
| Glyceryl triisostearate | 8 g |
| (DUB TGIS from Stéarineries Dubois) | |
| Magnesium stearate | 3 g |
| Paraffin wax microbeads | 4 g |
| MicroEase ® 114S from Micro Powders) | |
| Polyurethane and silica powder | 2 g |
| (Plastic Powder D400 from Toshiki) | |
| Hydrogenated polydecene | 8 g |
| (Ceraflow E ® from Shamrock) | |
| Preserving agents | 0.8 g |

The compacted composition shows good impact strength and is easy to erode using an applicator. The eyeshadow shows moderate sheen (at 45°), measured according to the protocol indicated above, of 1017±3.8% gloss units.

### EXAMPLE 2

The following compacted eyeshadow was prepared:

| | |
|---|---|
| Titanium oxide-coated alumina | 60 g |
| (Xirona Silver from Merck) | |
| Talc | 15.2 g |
| Glyceryl triisostearate | 7.5 g |
| (DUB TGIS from Stéarineries Dubois) | |
| Magnesium stearate | 3 g |
| Paraffin wax microbeads | 4 g |
| MicroEase ® 114S from Micro Powders) | |
| Polyurethane and silica powder | 2 g |
| (Plastic Powder D400 from Toshiki) | |
| Hydrogenated polydecene | 7.5 g |
| (Ceraflow E ® from Shamrock) | |
| Preserving agents | 0.8 g |

The compacted composition shows good impact strength and is easy to erode using an applicator. The eyeshadow shows moderate sheen (at 45°), measured according to the protocol indicated above, of 4267±9.9% gloss units.

1. A composition in compact powder form comprising:
a fatty phase comprising polydecene that is liquid at 25° C., at least one wax totally or partially in powder form, and at least one colouring agent chosen from diffractive pigments, interference pigments, reflective particles, and mixtures thereof, the composition having a sheen at 45° of greater than or equal to 1000 gloss units, or
a fatty phase comprising polydecene that is liquid at 25° C., at least one polyurethane powder and at least one colouring agent chosen from diffractive pigments, reflective particles, interference pigments, and mixtures thereof, the composition having a sheen at 45° of greater than or equal to 1000 gloss units, or
a fatty phase comprising polydecene that is liquid at 25° C. and at least 51% by weight of at least one colouring agent chosen from diffractive pigments, interference pigments, reflective pigments, and mixtures thereof relative to the total weight of the composition, or
a fatty phase comprising polydecene that is liquid at 25° C., the composition comprising less than 10% of colouring agent that produces a colour by absorption of at least part of the visible spectrum.

2. The composition according to claim 1, comprising a fatty phase comprising polydecene that is liquid at 25° C., at least one polyurethane powder and at least one colouring agent chosen from diffractive pigments, reflective particles, interference pigments, and mixtures thereof, the composition having a sheen at 45° of greater than or equal to 1000 gloss units.

3. The composition according to claim 1, comprising a fatty phase comprising polydecene that is liquid at 25° C. and at least 51% by weight of at least one colouring agent chosen from diffractive pigments, interference pigments and reflective pigments, and mixtures thereof relative to the total weight of the composition.

4. The composition according to claim 1, comprising a fatty phase comprising polydecene that is liquid at 25° C., the composition comprising less than 10% of colouring agent that produces a colour by absorption of at least part of the visible spectrum.

5. The composition according to claim 1, comprising a fatty phase comprising polydecene that is liquid at 25° C., at least one wax totally or partially in powder form, and at least one colouring agent chosen from diffractive pigments, interference pigments, reflective particles, and mixtures thereof, the composition having a sheen at 45° of greater than or equal to 1000 gloss units.

6. The composition according to claim 1, wherein the polydecene is present in the composition in a content ranging from 1% to 20% by weight relative to the total weight of the composition.

7. The composition according to claim 1, comprising or further comprising at least one diffractive pigment chosen from:
monolayer pigments comprising a reflective material chosen from metals and alloys thereof,
pigments with a multilayer structure comprising a layer of a reflective material chosen from metals and alloys thereof and also from non-metallic reflective materials, covered on at least one side with a layer of a dielectric material,
pigments composed of a preformed dielectric or ceramic material,
and mixtures thereof.

8. The composition according to claim 1, comprising or further comprising at least one interference pigment chosen from nacres, reflective interference particles and goniochromatic pigments, and mixtures thereof.

9. The composition according to claim 8, comprising or further comprising at least one goniochromatic pigment chosen from multilayer interference structures and liquid-crystal colouring agents.

10. The composition according to claim 8, comprising or further comprising at least one nacreous pigment chosen from titanium mica coated with an iron oxide, mica coated with bismuth oxychloride, titanium mica coated with chromium oxide, titanium mica coated with an organic dye, nacreous pigments based on bismuth oxychloride, mica particles at the surface of which are superposed at least two successive layers of metal oxides and/or of organic dyestuffs, and mixtures thereof.

11. The composition according to claim 8, comprising or further comprising at least one reflective interference particle chosen from particles with a synthetic substrate at least partially coated with at least one layer of at least one metal oxide.

12. The composition according to claim 1, wherein the reflective pigments are chosen from
metal oxides,
multilayer structures comprising a natural or synthetic substrate, at least partially coated with at least one layer of a reflective material, and
mixtures thereof.

13. The composition according to claim 1, wherein the colouring agent chosen from diffractive pigments, interference pigments, reflective particles, and mixtures thereof is present in a content ranging from 40% to 90% by weight relative to the total weight of the composition.

14. The composition according to claim 1, wherein the colouring agent chosen from diffractive pigments, interference pigments, reflective particles, and mixtures thereof is present in a content greater than or equal to 55% by weight relative to the total weight of the composition.

15. The composition according to any one of claims 2, 3 or 4, wherein it further comprises at least one wax.

16. The composition according to claim 15, wherein the wax is totally or partially in powder form.

17. The composition according to claim 15, wherein the wax is a paraffin wax and/or carnauba wax.

18. The composition according to claim 5, wherein the wax is present in a content ranging from 0.1% to 45% by weight relative to the total weight of the composition.

19. The composition according to claims 3, 4 or 5, further comprising a polyurethane powder.

20. The composition according to claim 19, wherein the polyurethane powder is a powdered copolymer of hexamethylene diisocyanate and of trimethylol hexyl lactone.

21. The composition according to claim 19, wherein the polyurethane powder is present in a content ranging from 0.1% to 45% by weight relative to the total weight of the composition.

22. The composition according to claim 1, further comprising at least one of a solid fatty phase and dry binder.

23. The composition according to claim 22, wherein the composition further comprises a solid fatty phase that comprises at least one compound chosen from waxes and metal soaps, and mixtures thereof.

24. The composition according to claim 22, wherein the composition further comprises a solid fatty phase in an amount of from 0.5% to 45% by weight relative to the total weight of the composition.

25. The composition according to claim 1, further comprising at least one compactable filler.

26. The composition according to claim 25, wherein the compactable filler is present in a content ranging from 0.05% to 40% by weight relative to the total weight of the composition.

27. The composition according to claim 1, further comprising at least one incompactable filler.

28. The composition according to claim 27, wherein the incompactable filler is present in a content ranging from 1% to 45% by weight relative to the total weight of the composition.

29. The composition according to claim 1, wherein the polydecene has a weight-average molecular weight ranging from 800 to 3000.

30. A process for making up an eyelid, comprising applying to an eyelid the composition according to claim 1.
